(19) United States
(12) Patent Application Publication
(10) Pub. No.: US 2008/0214958 A1
(43) Pub. Date: Sep. 4, 2008

(54) Blood Collection Apparatus

(75) Inventor: Edward Adamowicz, Derby (GB)

Correspondence Address: SMITH-HILL AND BEDELL, P.C., 16100 NW CORNELL ROAD, SUITE 220, BEAVERTON, OR, 97006, US

(73) Assignee: CINIMOD (IP) LIMITED, Burton-on-Trent (GB)

(21) Appl. No.: 11/916,696

(22) PCT Filed: Jun. 6, 2006
(86) PCT No.: PCT/GB2006/002080

§ 371 Date: Dec. 6, 2007

(30) Foreign Application Priority Data
Jun. 10, 2005 (GB) 0511791.6

(51) Int. Cl. A61B 5/15; A61B 1/303

(52) U.S. Cl. 600/581; 600/114; 600/583

(57) ABSTRACT

A blood collection apparatus for collecting a blood sample from a foetal scalp includes an amnioscope and a foetal scalp blood collection device. The amnioscope comprises an elongate tubular body for insertion into a vaginal canal, the elongate tubular body including guide means for guiding the foetal scalp blood collection device through the tubular body into position against a foetal scalp to enable the collection of a blood sample from the foetal scalp.

This is a national stage application filed under 35 USC 371 based on International Application No. PCT/GB2006/002080 filed Jun. 6, 2006, and claims priority under 35 USC 119 of United Kingdom Patent Application No. 0511791.6 filed Jun. 10, 2005.

Embodiments of the present invention relate to blood collection apparatus for collecting a blood sample from a foetal scalp and/or an amnioscope and/or a foetal scalp blood collection device.

Measurement of oxygen supply to a foetus can be desirable where there is concern that the foetus is not receiving an adequate supply of oxygen. This may be achieved by collecting blood from the foetal scalp and carrying out a blood gas analysis of the collected blood. Collection of blood is usually achieved by using a suitable blood collection device to incise the foetal scalp and collect blood from the incision, and an amnioscope may be used to facilitate insertion of the blood collection device into the vaginal canal.

According to a first aspect of the present, invention, there is provided blood collection apparatus for collecting a blood sample from a foetal scalp, the blood collection apparatus including an amnioscope and a foetal scalp blood collection device, the amnioscope comprising an elongate tubular body for insertion into a vaginal canal, the elongate tubular body including guide means for guiding the foetal scalp blood collection device through the tubular body into position against a foetal scalp to enable the collection of a blood sample from the foetal scalp.

According to a second aspect of the present invention, there is provided an amnioscope comprising an elongate tubular body for insertion into a vaginal canal, the elongate tubular body including guide means for guiding a foetal scalp blood collection device through the tubular body into position against a foetal scalp to enable the collection of a blood sample from the foetal scalp.

According to a third aspect of the present invention, there is provided a foetal scalp blood collection device adapted for use with an amnioscope to enable the collection of a blood sample from a foetal scalp, the device comprising a cutting member for incising a foetal scalp and a blood collector arranged to collect blood from an incised foetal scalp, the blood collection device being separable so that after collection of a blood sample the cutting member is adapted to be retained inside the amnioscope and the blood collector is adapted to be removed from the amnioscope.

The tubular body may define a distal end which may be adapted to be inserted into a vaginal canal and which may be locatable in use against a foetal scalp. The distal end may include a sealing arrangement for providing a seal between the distal end and the foetal scalp, in use. The sealing arrangement may comprise a generally annular seal which may be disposed on the distal end. The sealing arrangement may comprise a thermoplastic elastomer material, for example polyethylene, polythene, polypropylene or a thermoplastic rubber.

The tubular body may define a proximal end which may be adapted to remain outside the vaginal canal when the tubular body is located in the vaginal canal in use.

The elongate tubular body may define an inner surface and the guide means may be defined by the inner surface. The guide means may be elongate and may extend along the inner surface. The guide means may be generally coplanar with the inner surface and may extend between the proximal and distal ends of the tubular body.

The elongate tubular body may be generally conical, and may taper inwardly from the proximal end towards the distal end which may facilitate insertion of the amnioscope into a vaginal canal. The guide means may be defined in an in use lowermost region of the generally conical tubular body so that the guide means slopes generally downwardly with respect to the horizontal from the distal end towards the proximal end when the amnioscope is located in use in a vaginal canal.

The guide means may be arranged to guide a distal end of the blood collection device towards a central region of the distal end of the tubular body. The guide means may intersect the centerline of the tubular body at the distal end thereof.

The guide means may be co-operable in use with the blood collection device to control the movement of the blood collection device in the tubular body. The guide means may be co-operable in use with the blood collection device to prevent removal of the complete blood collection device from the tubular body after collection of a blood sample.

The guide means may comprise a recess formation which may be co-operable with a projection formation on the blood collection device to control the movement of the blood collection device in the tubular body. The recess formation may be co-operable with the projection formation to prevent said removal of the complete blood collection device from the tubular body after collection of a blood sample.

The recess formation may comprise an elongate groove, and the projection formation may include a resilient projection which may be locatable in the elongate groove.

The elongate groove may define proximal and distal ends and each of the proximal and distal ends may define a step with the adjacent surface of the guide means. The resilient projection may be arranged to abut the step at the proximal end of the elongate groove during movement of the blood collection device in a direction towards the proximal end of the tubular body which may prevent movement of the projection past the step and thereby prevent said removal of the complete blood collection device from the amnioscope.

The guide means may comprise a guide channel and the guide channel may be generally semi-cylindrical in cross-section.

The amnioscope may include a lighting arrangement which may be arranged to illuminate the distal end of the amnioscope and, in use, a foetal scalp. The tubular body may define a housing in which the lighting arrangement may be located, and the housing may be defined in an uppermost region of the tubular body when the tubular body is located in use in a vaginal canal.

The lighting arrangement may comprise a light source, for example a light emitting diode (LED), and may comprise an energy source, for example a battery. The amnioscope may include lighting control means for controlling the lighting arrangement. The lighting control means may comprise an isolator which may be locatable between the energy source and the light source to prevent the supply of energy to the light source. The isolator may comprise a non-electrically conductive member which may be flexible and which may be elongate. The isolator may be adapted for removal by a user from a location between the energy source and the light source to enable electrical energy to be provided by the energy source to the light source to activate the light source.

The amnioscope may comprise a protective member which may be adapted to protect a distal end of the blood collection device when the blood collection device is partially inserted into the amnioscope. The protective member may comprise a resiliently deformable portion which may be adapted to cover the distal end of the blood collection device when partially inserted into the amnioscope. The resiliently deformable portion may be adapted to be deflected by the blood collection device when fully inserted into the amnioscope. The protective member may be located on the guide means.

The blood collection device may comprise a cutting member which may be arranged to incise a foetal scalp, and may comprise a blood collector which may be arranged to collect blood from an incised foetal scalp. The blood collection device may be separable so that, after collection of a blood sample, the cutting member is adapted to be retained inside the amnioscope and the blood collector is adapted to be removed from the amnioscope.

The blood collection device may comprise first and second body portions and may be separable about an interface between the first and second body portions.

The first body portion may be rotatable relative to the second body portion which may provide for separation of the first and second body portions, for example by shear action. The second body portion may include anti-rotation means which may be co-operable in use with anti-rotation formations on the amnioscope which may prevent rotation of the second body portion relative to the amnioscope and which may permit rotation of the first body portion relative to the second body portion. The anti-rotation means may comprise an elongate groove which may be adapted to receive the anti-rotation formations on the amnioscope.

The blood collection device may include a blood detection system for detecting the presence of blood in the blood collector. The blood detection system may include indication means for indicating to a user when a sufficient quantity of blood has been collected from the foetal scalp. The indication means may comprise a visual indicator, for example a light emitting diode (LED), which may be adapted to illuminate in response to the detection of a predetermined quantity of blood in the blood collector. The indication means may comprise first and second visual indicators and may be arranged so that the first visual indicator is adapted to illuminate when a first predetermined quantity of blood is detected in the blood collector and so that the second visual indicator is adapted to illuminate when a second predetermined quantity of blood is detected in the blood collector.

The blood collection device may include switching means for activating the blood detection system and the switching means may be co-operable in use with the amnioscope upon insertion of the blood collection device into the amnioscope.

The switching means may be co-operable in use with the guide means to depress the switching means and thereby activate the blood detection system. The guide means may comprise a recess formation in which the switching means may be locatable in use so that the guide means may not be operable to depress the switching means when the blood collection device is located in predetermined positions in the amnioscope.

The blood collector may be elongate and may define proximal and distal ends. The blood collector may extend through the first and second body portions, and the distal end may extend from the second body portion so that it is located in use adjacent the foetal scalp to permit the collection of a sample of blood therefrom.

The first body portion may include holding means for holding the blood collector in position and may be arranged to hold the blood collector substantially adjacent the proximal end thereof. The holding means may be arranged to clamp the blood collector substantially adjacent the proximal end thereof, such that the proximal end is preferably open to atmosphere.

The blood collection device may define a distal end which may be provided on the second body portion and which may be locatable in use against a foetal scalp. The distal end may include a sealing arrangement which may be adapted to provide a seal between the distal end of the blood collection device and a foetal scalp, in use. The sealing arrangement may comprise a generally annular seal. The sealing arrangement may comprise a thermoplastic elastomer material, for example polyethylene, polythene, polypropylene or a thermoplastic rubber.

The blood collection device may include a biasing arrangement which may be operable to urge the cutting member from a primed condition to a cutting condition in which it may operable to incise a foetal scalp. The blood collection device may include a retaining arrangement for retaining the cutting member in the primed condition and may include a trigger arrangement movable between a first condition in which it may be adapted to hold the retaining arrangement in a retaining condition and a second condition in which it may be adapted to allow movement of the retaining arrangement from the retaining condition to a release condition.

The trigger arrangement may be locatable in use against a foetal scalp and may be arranged to be pressed against the foetal scalp to move the trigger arrangement from the first condition to the second condition. The blood collection device may include a latch arrangement which may be adapted to permit movement of the trigger arrangement from the first condition to the second condition only upon the application of a minimum predetermined force against the foetal scalp. The latch arrangement may include a pin which may be locatable in a corresponding slot which may be elongate. The slot may be provided on the trigger arrangement and may include opposing inwardly directed projections. The latch arrangement may be adapted to prevent movement of the trigger arrangement from the first condition to the second condition by preventing movement of the pin past the inwardly directed projections. The latch arrangement may be arranged to permit movement of the pin past the inwardly directed projections upon the application of said minimum predetermined force against the foetal scalp using the trigger arrangement.

An embodiment of the present invention will now be described by way of example only and with reference to the accompanying drawings, in which:—

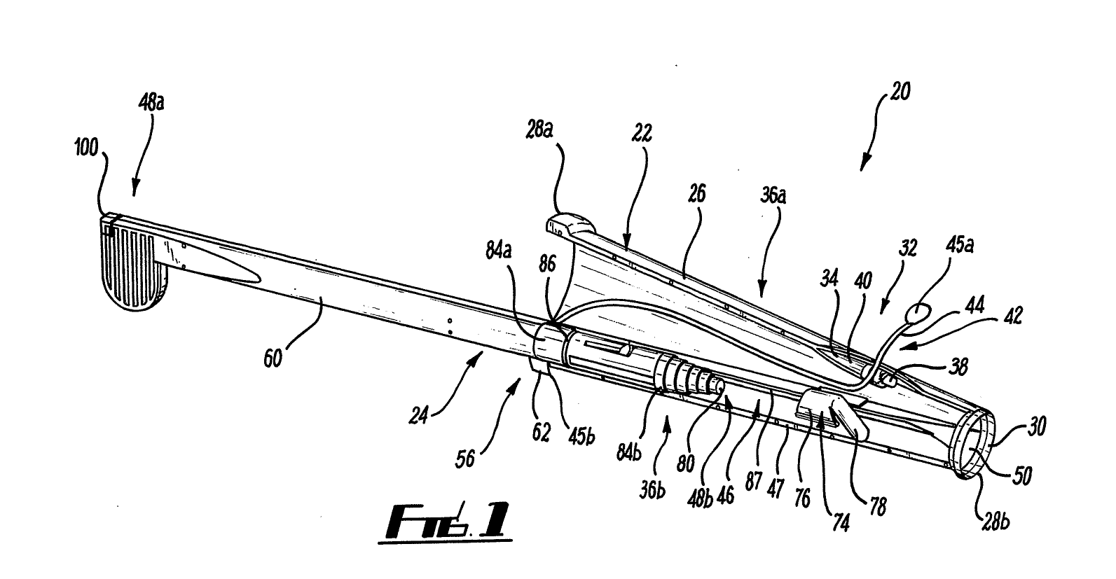

FIG. 1 is a partially cut away diagrammatic perspective view of a blood collection apparatus in a first condition;

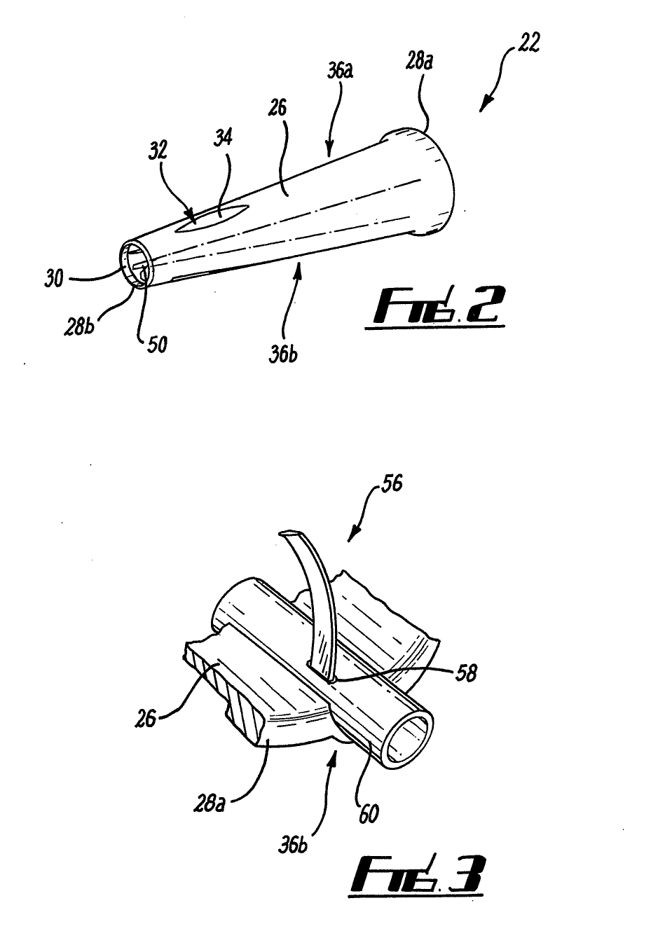

FIG. 2 is a diagrammatic perspective view of an amnioscope of the blood collection apparatus of FIG. 1;

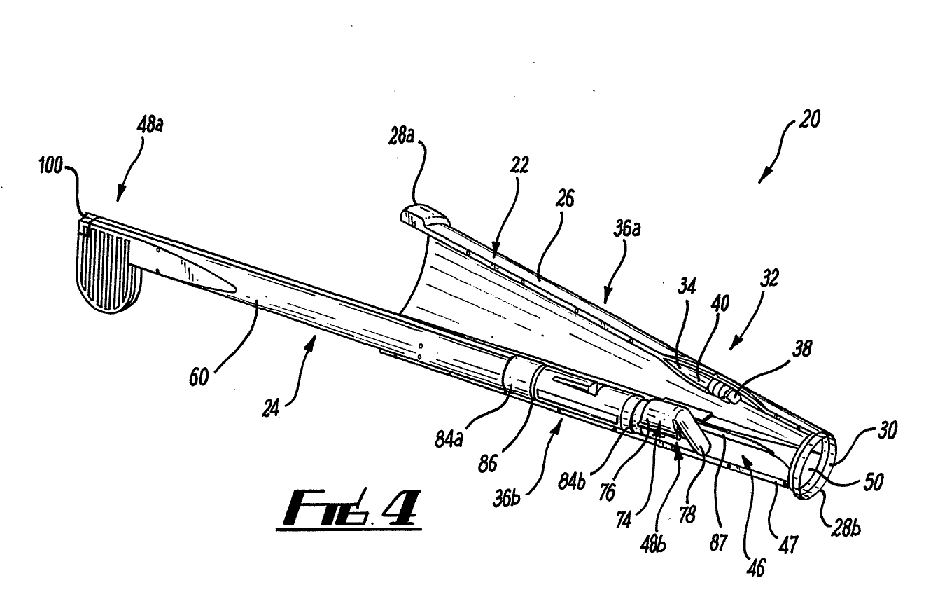

FIG. 3 is an enlarged perspective view of part of a proximal end of the amnioscope;

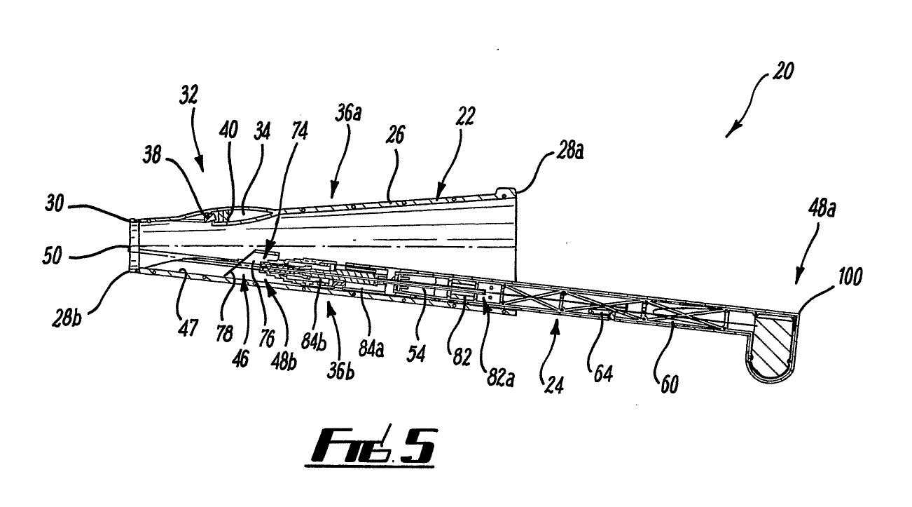

FIG. 4 is a partially cut away diagrammatic perspective view of the blood collection apparatus of FIG. 1 in a second condition;

FIG. 5 is a diagrammatic cross-sectional side view of the blood collection apparatus of FIGS. 1 and 4 in the second condition;

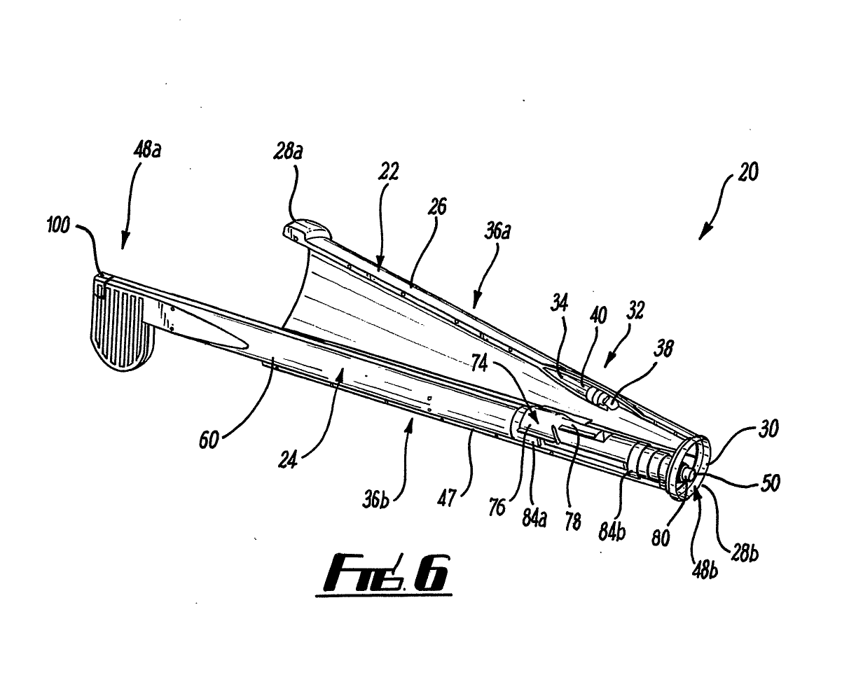

FIG. 6 is a partially cut away diagrammatic perspective view of the blood collection apparatus of FIGS. 1, 4 and 5 in a third condition;

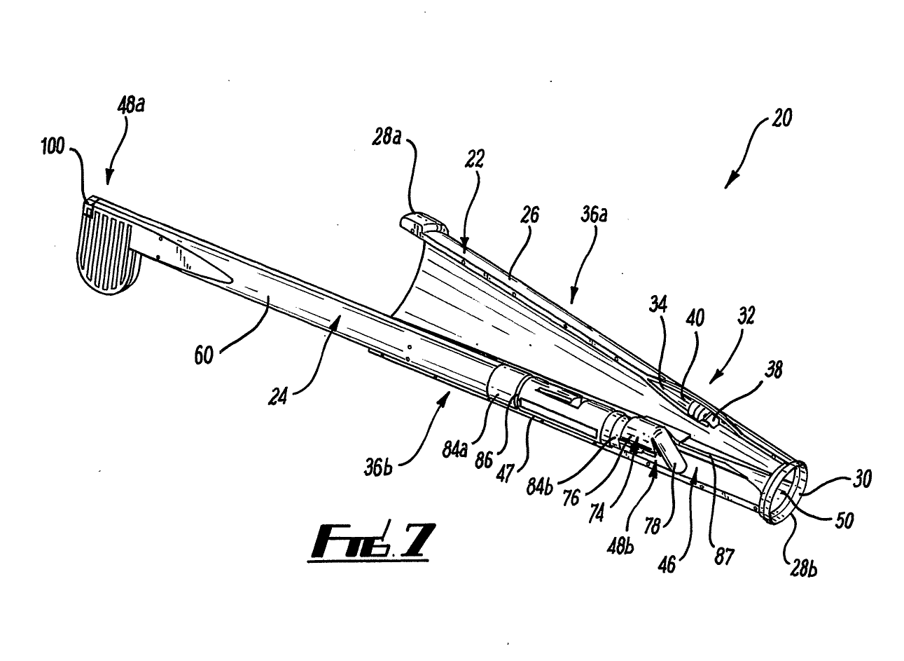

FIG. 7 is a partially cut away diagrammatic perspective view of the blood collection apparatus of FIGS. 1 and 4 to 6 in a fourth condition;

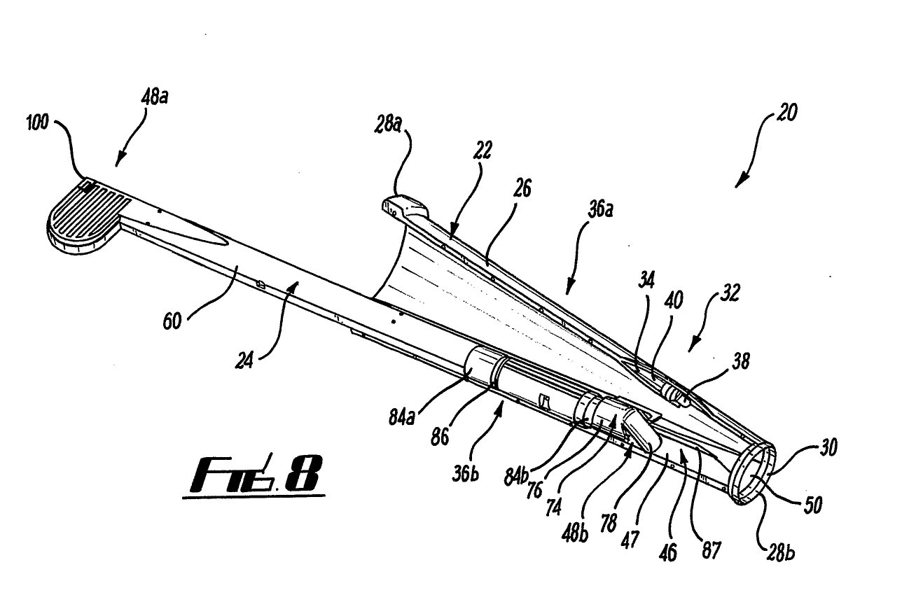

FIG. 8 is a partially cut away diagrammatic perspective view of the blood collection apparatus of FIGS. 1 and 4 to 7 in a fifth condition;

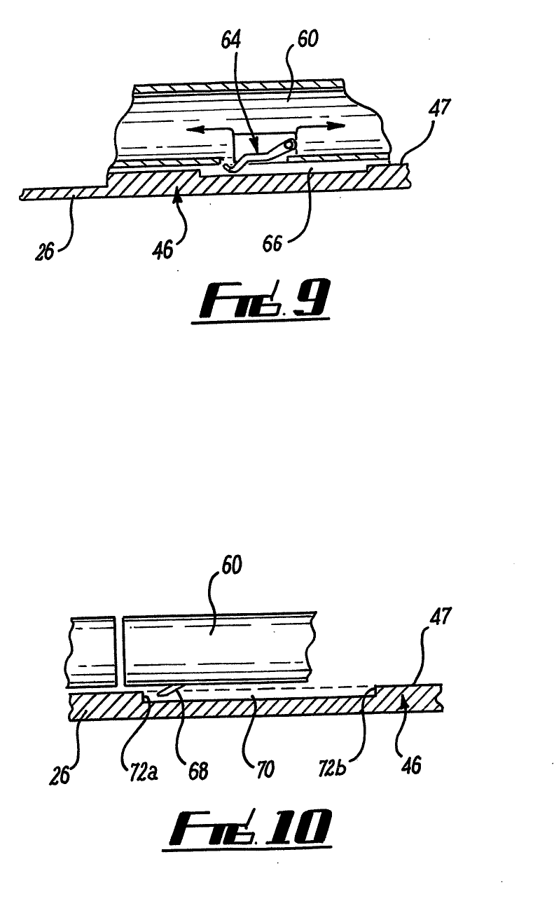

FIGS. 9 and 10 are enlarged diagrammatic cross-sectional views of parts of the blood collection apparatus of FIGS. 1 and 4 to 8;

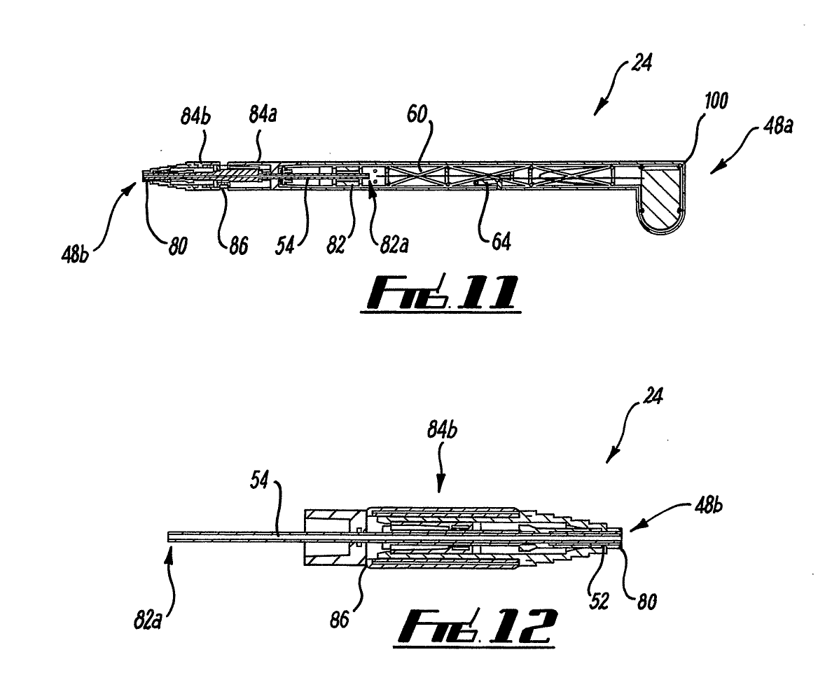

FIG. 11 is a diagrammatic cross-sectional view of a blood collection device of the blood collection apparatus of FIGS. 1 and 4 to 8;

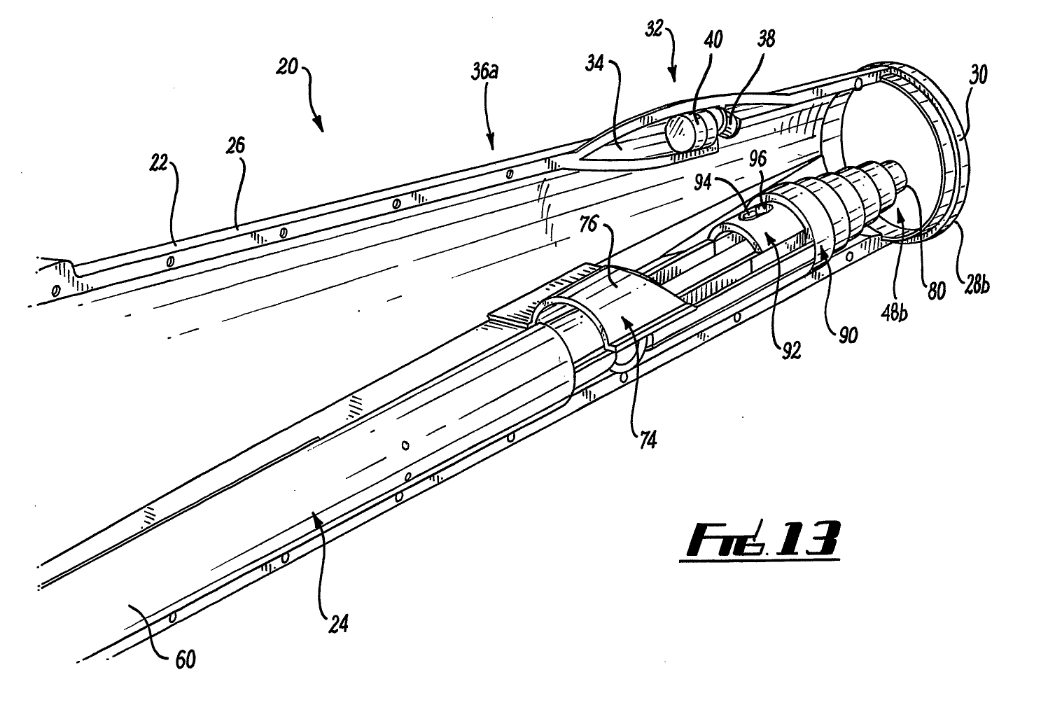

FIG. 12 is a diagrammatic cross-sectional view of a second body portion of the blood collection device of FIG. 11;

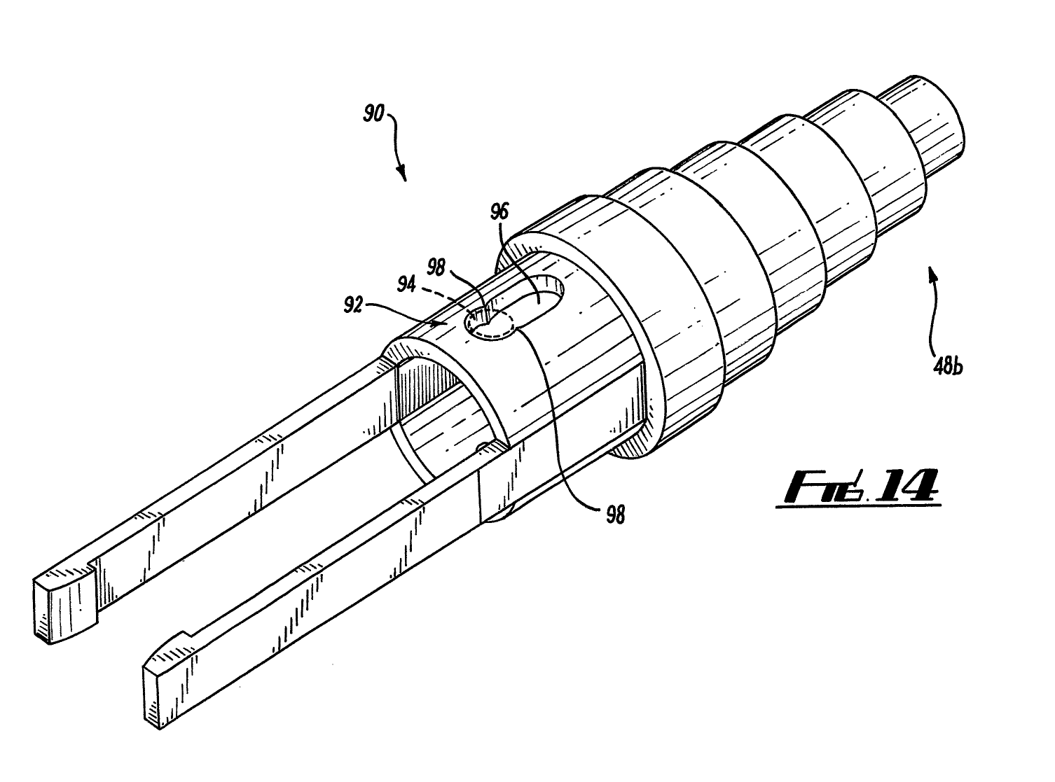

FIG. 13 is a partially cut away diagrammatic perspective view of a distal end of the blood collection apparatus;

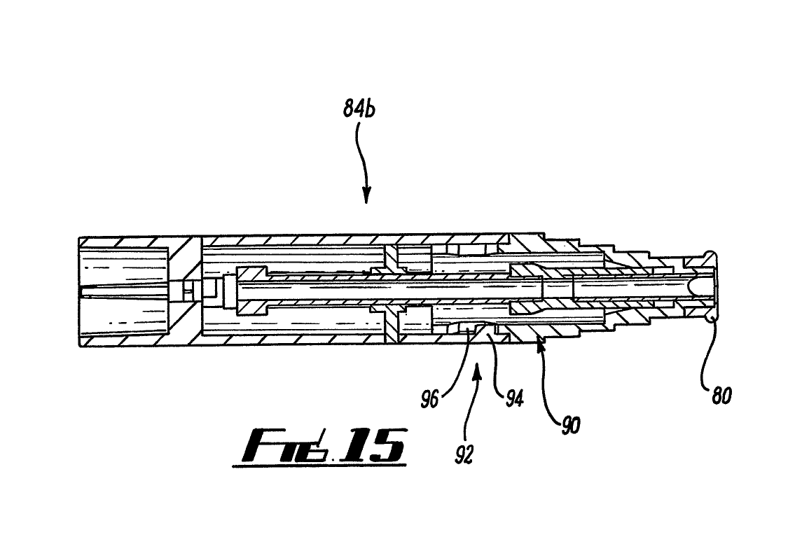

FIG. 14 is an enlarged view of a trigger arrangement of the blood collection device; and

FIG. 15 is a further diagrammatic cross-sectional view of the second body portion of the blood collection device of FIG. 11.

Referring to the drawings, there is shown generally blood collection apparatus 20 for collecting a blood sample from a foetal scalp (not shown). The blood collection apparatus 20 comprises an amnioscope 22 and a foetal scalp blood collection device 24.

The basic principle of operation of the foetal scalp blood collection device 24 has already been described in the applicant's earlier published international patent application no. WO 2004/098410 A1, the content of which is incorporated herein in its entirety.

In more detail, the amnioscope 22 comprises a generally elongate tubular body 26 which is inserted in use into a vaginal canal of a female patient. The tubular body 26 defines proximal and distal ends 28a, 28b, and tapers from the proximal end 28a towards the distal end 28b to define a conical shape which facilitates insertion of the tubular body 26 into a vaginal canal. When the tubular body 26 is fully inserted into a vaginal canal in use, the distal end 28b abuts the foetal scalp from which the blood sample is to be collected whilst the proximal end 28a remains outside the vaginal canal.

A sealing arrangement in the form of an annular seal 30 is provided on the distal end 28b of the tubular body 26. The annular seal 30 ensures that a seal is established between the distal end 28b of the tubular body 26 and the foetal scalp and thus prevents contamination of the blood sample during collection by preventing the entry of amniotic fluid, meconium and the like into the tubular body 26. The annular seal 30 comprises a suitably compliant material, such as a thermoplastic elastomer material, and is capable of maintaining a seal between the tubular body 26 and the foetal scalp in the event of relative movement between the amnioscope 22 and the foetal scalp. This may occur, for example, as a result of movement of the foetus, movement the patient, or movement of the amnioscope 22 by the user.

In order to facilitate observation of the foetal scalp through the tubular body 26, the amnioscope 22 includes an integrated lighting arrangement 32 which is located in a housing 34 defined in the tubular body 26 in an in use uppermost region 36a. The uppermost region 36a is that region of the tubular body 26 which is uppermost when the tubular body 26 is located in use in a vaginal canal.

The lighting arrangement 32 comprises a light source, such as a light emitting diode (LED) 38, which is positioned so that it directs light towards the distal end 28b of the inside of the tubular body 26, and an energy source, such as a battery 40, which is operable to provide electrical energy to the LED 38 to illuminate the LED 38.

In order to enable the lighting arrangement 32 to be controlled, and in particular to be activated prior to insertion of the tubular body 26 into a vaginal canal, the amnioscope 22 includes lighting control means 42 in the form of an isolator 44. As best seen in FIG. 1, the isolator 44 comprises a flexible elongate non-electrically conductive member which is locatable between the battery 40 and the LED 38 to isolate them and prevent the supply of electrical energy from the battery 40 to the LED 38.

In order to activate the LED 38, a user must remove the isolator 44 by grasping an end 45a thereof and pulling the end 45a so that the isolator 44 is removed from its location between the battery 40 and LED 38, thereby enabling electrical contact to be established therebetween.

The tubular body 26 includes guide means 46 arranged to guide the blood collection device 24 through the tubular body 26 into position against a foetal scalp to enable the collection of a blood sample from the foetal scalp.

The guide means 46 is defined in an inner surface 47 of the tubular body 26 and is coplanar with the inner surface 26. The guide means 46 is elongate and extends along the inner surface 47 between the proximal and distal ends 28a, 28b. In a preferred embodiment of the invention, the guide means 46 is in the form of a guide channel which is generally semi-cylindrical in cross-section.

The guide means 46 is defined in a lowermost region 36b of the tubular body 26 diametrically opposite to the location of the housing 34, and slopes generally downwardly with respect to the horizontal from the distal end 28b towards the proximal end 28a of the tubular body 26. This ensures that the blood collection device 24 also slopes downwardly in the same direction, in use, thereby facilitating collection of a blood sample from the foetal scalp.

The blood collection device 24 includes a distal end 48b and a proximal end 48a, and the guide means 46 is arranged to guide the distal end 48b towards a central region 50 at the distal end 28b of the tubular body 26. This enables a user to easily identify the location on the foetal scalp that will be incised by the blood collection device 24 when inserted into the tubular body 26.

Referring to FIG. 12, and as described in detail in WO 2004/098410 A1, the blood collection device 24 comprises a cutting member 52 for incising the foetal scalp, and a biasing arrangement (not shown) which is operable to urge the cutting member 52 from a primed condition to a cutting condition, and from the cutting condition to a retracted condition. The blood collection device 24 also includes a blood collector in the form of a capillary tube 54 which is arranged to collect blood from an incised foetal scalp.

The blood collection apparatus 10 is initially supplied to an end user, such as a medical practitioner, in a first condition, as illustrated in FIG. 1. In this first condition, the cutting member 52 of the blood collection device 24 is in the primed condition, ready to incise a foetal scalp, and the blood collection device 24 is located in a partially inserted position in the tubular body 26, part way along the guide means 46.

In order to prevent accidental movement of the blood collection device 24 into the amnioscope 22, the blood collection apparatus 10 includes stop means 56 defined by the isolator 44 to retain the blood collection apparatus 10 in the first condition. As best seen in FIGS. 1 and 3, the isolator 44 extends from the lighting arrangement 32 through the interior of the tubular body 26 and through apertures 58 in a handle 60 of the blood collection device 24 and in the tubular body 26 adjacent the proximal end 28a thereof.

As indicated above, the isolator 44 is fabricated from a flexible material which nevertheless has sufficient rigidity to prevent a user from pushing the blood collection device 24 into the tubular body 26 beyond the position shown in FIG. 1. A removable tab 62 is provided on an end 45b of the isolator 44 and this must be removed by a user before the isolator 44 can be removed by pulling on the opposite end 45a, as already explained above.

In order to enable the blood collection apparatus 10 to be used, the isolator 44 is firstly removed by initially removing the tab 62 and then pulling on the end 45a. This causes activation of the lighting arrangement 32, as explained above. The amnioscope 22 is then inserted into a vaginal canal until the distal end 28b of the tubular body 26 is pressed against the foetal scalp, the viewing of which is facilitated by the activated lighting arrangement 32.

As the blood collection device 24 has not yet been moved from the position shown in FIG. 1, a user is provided with a clear view of the illuminated region of the foetal scalp through the tubular body 26. Pressing the distal end 28b against the foetal scalp establishes a seal between the distal end 28b and the foetal scalp by way of the annular seal 30.

Once the distal end 28b of the tubular body 26 is in position against the foetal scalp, the blood collection device 24 is advanced into the tubular body 26 along the guide means 46. This is achieved by pushing the blood collection device 24 into the tubular body 26 using the handle 60.

The blood collection device 24 includes switching means in the form of a spring loaded microswitch 64 (see FIG. 5) which extends from the handle 60 and which is operable to activate a blood detection system of the blood collection device 24 by co-operating with the inner surface 47 of the tubular body 26. The blood detection system comprises an infrared emitter and detector arrangement and has already been described in detail in WO 2004/098410 A1.

When the blood collection device 24 has been pushed into the tubular body 26 to the position shown in FIG. 6, the microswitch 64 engages the inner surface 47 along the guide means 46 and is depressed, thereby activating the blood detection system. As illustrated in FIG. 9, the guide means 46 includes an elongate recess formation 66 which extends part way along the guide means 46 and in which the microswitch 64 is locatable. The microswitch 64 is located in the recess formation 66 until the blood collection device 24 is pushed into the tubular body 26 to the position shown in FIG. 6, in which the blood collection apparatus 10 is in the third condition.

Referring to FIG. 10, the handle 60 of the blood collection device 24 includes a resilient projection 68 which is locatable in a further recess formation, in the form of an elongate groove 70, in the guide means 46. The groove 70 defines steps 72a, 72b at each end with the adjacent surface of the guide means 46. As the blood collection device 24 is pushed into the tubular body 26 from the position shown in FIG. 1 to the position shown in FIG. 4, the inherent resilience of the projection 68 causes it to snap into the groove 70.

Due to the direction in which the projection 68 extends from the handle 60, namely in a direction from the distal end 48b towards the proximal end 48a of the blood collection device to define an acute angle with the handle 60, the projection 68 abuts the step 72a if removal of the blood collection device 24 from the tubular body 26 is attempted, thereby preventing said removal. The projection 68 is however capable of riding over the step 72b to allow the blood collection device 24 to be pushed further into the tubular body 26, as will be clearly appreciated from FIG. 10.

It may be desirable to clean the region of the foetal scalp from which blood is to be collected prior to collecting a blood sample, and a suitable known disposable cleaning swab may be passed down the tubular body 26 for this purpose. Cleaning is usually effected when the blood collection apparatus 10 is in the second condition, as illustrated in FIGS. 4 and 5.

The amnioscope 22 includes a protective member 74 which is adapted to cover and protect the distal end 48b of the blood collection device 24 when the blood collection apparatus 10 is in the second condition, as illustrated in FIGS. 4 and 5. The protective member 74 is mounted on the guide means 46 and comprises a semi-cylindrical portion 76 having a resilient finger 78 extending therefrom, the inherent resilience of the finger 78 biasing it downwardly towards the guide means 46. In this downwardly biased position, the resilient finger 78 covers the distal end 48b of the blood collection device 24 thereby preventing contamination of the capillary tube 54, for example during swabbing of the foetal scalp prior to blood collection.

In order to enable the collection of a blood sample from a foetal scalp, the blood collection device 24 is pushed further into the tubular body 26, along the guide means 46, to the position shown in FIG. 6, in which the distal end 48b of the blood collection device 24 is pressed against the foetal scalp. The blood collection apparatus 10 is now in the third condition.

As the blood collection device 24 is pushed into the tubular body 26, it passes through an opening defined between the semi-cylindrical portion 76 of the protective member 74 and the guide means 46, and deflects the resilient finger 78 upwardly to the position shown in FIG. 6.

Once the distal end 48b of the blood collection device 24 is pressed against the foetal scalp, the application of further force, using the handle 60, actuates the blood collection device 24 by moving a trigger arrangement 90 from a first condition to a second condition in which it allows the biasing arrangement to urge the cutting member 52 from the primed condition to the cutting condition. The biasing arrangement subsequently moves the cutting member 52 from the cutting condition to the retracted condition, and blood is then collected in the capillary tube 54, as explained in WO 2004/098410 A1.

The distal end 48b of the blood collection device 24 includes a sealing arrangement 80 which is operable to provide a seal between the distal end 48b and the foetal scalp to facilitate the collection of a blood sample. The sealing arrangement 80 comprises an annular seal which, like the annular seal 30, comprises a suitable thermoplastic elastomer material. Like the annular seal, the sealing arrangement 80 allows for relative movement between the blood collection device 24 and the foetal scalp whilst maintaining an adequate seal.

In order to prevent accidental actuation of the blood collection device 24, the blood collection device 24 includes a latch arrangement 92 which permits movement of the trigger arrangement 90 from the first condition to the second condition, and hence actuation of the blood collection device 24, only upon the application of a minimum predetermined force against the foetal scalp. As seen in FIGS. 13 to 15, the latch arrangement 92 comprises a pin 94 (shown in broken lines in FIGS. 13 and 14) which is defined by the second body portion 84b of the blood collection device 24. The pin 94 is locatable in an elongate slot 96 which is provided on the trigger arrangement 90 and which has inwardly directed projections 98.

The pin 94 engages the inwardly directed projections 98, as shown in FIG. 14, to prevent movement of the trigger arrangement 90 relative to the second body portion 84b from the first condition to the second condition. Upon the application of a minimum predetermined force against the foetal scalp, movement of the pin 94, from the position shown in FIG. 14, along the slot 96 past the inwardly directed projections 98, and hence movement of the trigger arrangement 90 from the first condition to the second condition, is possible as a result of deformation of the inwardly directed projections 98 by the pin 94.

The collection of blood from the incised foetal scalp into the capillary tube 54 is monitored by the blood detection system. The blood detection system includes visual indication means 100 in the form of first and second light emitting diodes (LEDs) for providing to a user an indication of the progress of blood collection. The first LED may be connected to a first infrared detector and emitter arrangement located in a first position along the capillary tube 54 such that the first LED illuminates to indicate that a minimum predetermined quantity of blood has been collected in the capillary tube 54. The second LED may be connected to a second infrared detector and emitter arrangement located in a second position along the capillary tube 54, closer to the proximal end thereof, such that the second LED illuminates to indicate that an optimum predetermined quantity of blood has been collected in the capillary tube 54.

As illustrated in FIG. 11, the capillary tube 54 is mounted in the handle 60 using a holding means 82 which holds the capillary tube 54 adjacent the proximal end 82a thereof. The proximal end 82a is, however, open to atmosphere to promote the collection of blood into the capillary tube 54. The holding means 82 may be mounted in different positions inside the handle 60 to enable the capillary tube 54 to be mounted at different positions relative to the infrared emitter and detector arrangements. This enables the operation the blood detection system to be easily modified by simple repositioning of the capillary tube 54 so that different amounts of blood can collected before the first and second LEDs are illuminated.

After the LEDs have illuminated to indicate that a sufficient quantity of blood has been collected in the capillary tube 54, the blood collection device 24 is retracted along the guide means 46 from the position shown in FIG. 6 to the position shown in FIG. 7, in which the blood collection apparatus 10 is in a fourth condition. In this position, which is substantially the same as the position illustrated in FIG. 4, the resilient finger 78 returns to its original position due to its inherent resilience and thus again covers and protects the distal end 48b of the blood collection device 24.

As the blood collection device 24 is retracted along the guide means 46, a position will be reached at which further withdrawal of the blood collection device 24 is not possible. This is caused by abutment of the projection 68 with the step 72a.

In order to enable the capillary tube 54 containing the collected blood sample to be removed from the blood collection apparatus 10 to enable the sample to be analysed, the blood collection device 24 comprises first and second body portions 84a, 84b which define therebetween an interface 86 about which the first and second body portions 84a, 84b are rotatable relative to each other to enable them to be separated. Relative rotation of the first and second body portions 84a, 84b about the interface 86 causes the first and second body portions 84a, 84b to separate by shear action.

The first body portion 84a is formed integrally with the handle 60, and hence rotation of the handle 60, for example to the position shown in FIG. 8, causes the first body portion 84a to rotate. Rotation of the second body portion 84b is prevented due to the engagement of anti-rotation formations 87 on the tubular body 26 in grooves on opposite sides of the blood collection device 24.

The handle 60 is connected to the blood collection device 24 via the first body portion 84a. As the handle 60 holds the capillary tube 56, separation of the first body portion 84a from the second body portion 84b results in separation of the handle 60, and hence the capillary tube 56, from the second body portion 84b. As the second body portion 84b, which houses the cutting member 52, remains securely inside the tubular body 26, the cutting member 52 is completely inaccessible and hence there is no risk of injury. After use, the amnioscope 22 with the second body portion 84b retained inside may be disposed.

There is thus provided a blood collection apparatus 20 which provides a simple and effective means for collecting a blood sample from a foetal scalp.

Although embodiments of the invention have been described in the preceding paragraphs with reference to various examples, it should be appreciated that various modifications to the examples given may be made without departing from the scope of the present invention. For example, the tubular body 26 may be of any suitable shape or configuration. The first and second body portions 84a, 84b may be separable other than by shear action. The guide means 46 may be of any suitable shape or configuration. The blood detection system may comprise a single infrared emitter and detector arrangement which may be connected to a visual indication means 100 in the form of a single LED.

Whilst endeavouring in the foregoing specification to draw attention to those features of the invention believed to be of particular importance, it should be understood that the Applicant claims protection in respect of any patentable feature or combination of features hereinbefore referred to and/or shown in the drawings, whether or not particular emphasis has been placed thereon.

1-59. (canceled)

60. Blood collection apparatus for collecting a blood sample from a foetal scalp, the blood collection apparatus including an amnioscope and a foetal scalp blood collection device, the amnioscope comprising an elongate tubular body for insertion into a vaginal canal, the elongate tubular body including guide means for guiding the foetal scalp blood collection device through the tubular body into position against a foetal scalp to enable the collection of a blood sample from the foetal scalp.

61. Blood collection apparatus according to claim 60, wherein the tubular body defines a distal end which is adapted to be inserted into a vaginal canal and which is locatable in use against a foetal scalp.

62. Blood collection apparatus according to claim 61, wherein the guide means is arranged to guide a distal end of the blood collection device towards a central region of the distal end of the tubular body.

63. Blood collection apparatus according to claim 60, wherein the guide means is co-operable in use with the blood collection device to control the movement of the blood collection device in the tubular body.

64. Blood collection apparatus according to claim 63, wherein the guide means is co-operable in use with the blood collection device to prevent removal of the complete blood collection device from the tubular body after collection of a blood sample.

65. Blood collection apparatus according to claim 63, wherein the guide means comprises a recess formation which is co-operable with a projection formation on the blood collection device to control the movement of the blood collection device in the tubular body.

66. Blood collection apparatus according to claim 65, wherein the recess formation is co-operable with the projection formation to prevent said removal of the complete blood collection device from the tubular body after collection of a blood sample.

67. Blood collection apparatus according to claim 65, wherein the recess formation comprises an elongate groove, and the projection formation includes a resilient projection which is locatable in the elongate groove.

68. Blood collection apparatus according to claim 67, wherein the elongate groove defines proximal and distal ends and each of the proximal and distal ends define a step with an adjacent surface of the guide means.

69. Blood collection apparatus according to claim 68, wherein the resilient projection is arranged to abut the step at the proximal end of the elongate groove during movement of the blood collection device in a direction towards the proximal end of the tubular body to prevent movement of the projection past the step and thereby prevent said removal of the complete blood collection device from the amnioscope.

70. Blood collection apparatus according to claim 61, wherein the amnioscope includes a lighting arrangement which is arranged to illuminate the distal end of the tubular body and, in use, a foetal scalp.

71. Blood collection apparatus according to claim 70, wherein the tubular body defines a housing in which the lighting arrangement is located, the housing being defined in an uppermost region of the tubular body when the tubular body is located in use in a vaginal canal.

72. Blood collection apparatus according to claim 70, wherein the lighting arrangement includes a light source and an energy source and the amnioscope includes lighting control means for controlling the lighting arrangement.

73. Blood collection apparatus according to claim 72, wherein the lighting control means comprises an isolator locatable between the energy source and the light source to prevent the supply of energy to the light source, the isolator being adapted for removal by a user from a location between the energy source and the light source to enable electrical energy to be provided by the energy source to the light source to activate the light source.

74. Blood collection apparatus according to claim 60, wherein the blood collection device comprises a cutting member which is arranged to incise a foetal scalp, and a blood collector which is arranged to collect blood from an incised foetal scalp.

75. Blood collection apparatus according to claim 74, wherein the blood collection device is separable so that, after collection of a blood sample, the cutting member is adapted to be retained inside the amnioscope and the blood collector is adapted to be removed from the amnioscope.

76. Blood collection apparatus according to claim 74, wherein the blood collection device includes a blood detection system for detecting the presence of blood in the blood collector, the blood detection system including indication means for indicating to a user when a sufficient quantity of blood has been collected from the foetal scalp.

77. Blood collection apparatus according to claim 74, wherein the blood collection device includes a biasing arrangement operable to urge the cutting member from a primed condition to a cutting condition in which it is operable to incise a foetal scalp.

78. Blood collection apparatus according to claim 77, wherein the blood collection device includes a retaining arrangement for retaining the cutting member in the primed condition and a trigger arrangement movable between a first condition in which it is adapted to hold the retaining arrangement in a retaining condition and a second condition in which it is adapted to allow movement of the retaining arrangement from the retaining condition to a release condition.

79. Blood collection apparatus according to claim 78, wherein the trigger arrangement is locatable in use against a foetal scalp and is arranged to be pressed against the foetal scalp to move the trigger arrangement from the first condition to the second condition.
